(19) United States
(12) Patent Application Publication
(10) Pub. No.: US 2022/0130487 A1
(43) Pub. Date: Apr. 28, 2022

(54) DRUG VIRTUAL SCREENING SYSTEM FOR CRYSTAL COMPLEXES, AND METHOD OF USING THE SAME

(71) Applicant: SHENZHEN JINGTAI TECHNOLOGY CO., LTD., Gbuangdong (CN)

(72) Inventors: Lijun YANG, Guangdong (CN); Min XU, Guangdong (CN); Peiyu ZHANG, Guangdong (CN); Jian MA, Guangdong (CN); Shuhao WEN, Guangdong (CN); Lipeng LAI, Guangdong (CN)

(73) Assignee: SHENZHEN JINGTAI TECHNOLOGY CO., LTD., Guangdong (CN)

(21) Appl. No.: 17/427,103

(22) PCT Filed: Jun. 28, 2020
(86) PCT No.: PCT/CN2020/098530

§ 371 Date: Jul. 30, 2021

(51) Int. Cl. G16B 15/30; G06N 3/08

(52) U.S. Cl. CPC G16B 15/30; G06N 3/08

(57) ABSTRACT

The present invention provides a drug virtual screening system for crystal complexes, and method of using the same, comprising a visualization subsystem, an evaluation tool box subsystem, an AI model management subsystem, a large-scale sampling subsystem, a virtual screening subsystem, and a data log storage subsystem. Starting with the known crystal complexes, a batch of candidate compounds that meet the requirements are recommended after going through the visualization subsystem, evaluation tool box subsystem, AI model management subsystem, large-scale sampling subsystem, and virtual screening system in turn. Based on this system, the generation of the compound library is organically combined with the subsequent virtual screening. Users only need to describe the action mode of the drug on the protein and the requirements for the drug to generate a batch of compounds that meet the expectations. The automated system reduces user intervention and improves the efficiency of research and development.

### BACKGROUND OF THE INVENTION

### 1. Technical Field

This application pertains to the technical field of computer-aided drug design, in particular to a virtual drug screening system involving crystal complexes and method.

### 2. Background of Related Art

In traditional drug research and development, after obtaining crystal complexes of drugs and proteins in early high-throughput screening, the action mode is analyzed, and the structure of existing compounds is replaced to obtain new compounds based on the principle of bioelectronics isometrics and drug design experience. Traditional research and development methods include: bioelectronic isostere replacement, molecular docking, scaffold hopping, and virtual screening.

Generally speaking, these technologies are already available in common drug design software including MOE, Maestro, Discovery Studio to meet the needs of conventional drug research and development.

However, with the development of current medicinal chemistry theories and organic chemistry synthetic methods, when a potential compound is discovered, pharmaceutical research institutions usually conduct in-depth research on possible substituent groups, synthesize and test the activity of the derivatives, and finally get a fully perfect structure-activity relationship. This makes it almost impossible for subsequent researchers to obtain new drugs with the same scaffold.

Drug patents take into account traditional new drug design strategies, and will protect the structures of compounds that may be obtained by applying traditional drug design strategies, making it difficult for latecomers to obtain new drugs through simple substitutions.

Traditional methods such as molecular docking and pharmacophore models rely heavily on the selected compound library. The current compound library usually has several hundreds of thousands of molecules. The compound library released for many years has been explored many times by predecessors. The number of compounds is small and it is difficult to have a novel scaffold. Using AI-generated compounds can produce hundreds of thousands of compounds at one time, which has a broader space for exploration.

### SUMMARY OF THE INVENTION

In view of the above technical problems, the purpose of the present invention is to provide a virtual drug screening system for crystal complexes. This method can effectively solve the problem of traditional new drug design strategies that are difficult to obtain new scaffolds and break the barriers of existing compound patents. At the same time, the generated compound library is more target-specific than traditional compound libraries.

In order to achieve the above objective, the technical solution of the present invention is as follows:

A virtual drug screening system for crystal complexes, including: a visualization subsystem, an evaluation tool box subsystem, an AI model management subsystem, a large-scale sampling subsystem, a virtual screening subsystem, and a data log storage subsystem; Starting with the known crystal complexes, a batch of candidate compounds that meet the requirements are recommended after going through the visualization subsystem, evaluation tool box subsystem, AI model management subsystem, large-scale sampling subsystem, and virtual screening system in turn.

The visualization subsystem is used to view the binding position of the ligand in the protein in the crystal complex, analyze the binding mode of the ligand and the protein, and extract features that enhance the affinity of the drug to the protein.

The evaluation tool box subsystem encapsulates a plurality of compound evaluation modules, and is used to design an evaluation function by selecting a plurality of compound evaluation modules and assigning appropriate weights;

The AI model management subsystem is used for AI model, AI model training and AI model parameter update;

The large-scale sampling subsystem is used to sample and screen the trained AI model to obtain a compound library composed of corresponding compounds;

The virtual screening subsystem is used for further screening of compounds in the compound library;

The data log storage subsystem is used to establish and store a user's log information file; the log information file is used to record user operation records and generate corresponding data.

The present invention adopts the above technical solution, and its advantage is that the user can define the key characteristics of the drug by analyzing the binding mode of the ligand in the crystal complex, and set the physical and chemical properties that the candidate compound should have. The AI model updates the parameters according to user-defined requirements, and generates a batch of compounds that meet the conditions. These compounds are sorted into a compound library after conditional filtering. Virtually screen the compounds in the compound library, and finally get a batch of candidate compounds. The functional structure and flow of the system are shown in FIG. 1.

Preferably, the feature of enhancing the affinity of the drug to the protein is hydrogen bonding and/or hydrophobic interaction.

Preferably, the evaluation function is a weighted arithmetic mean, a weighted geometric mean, or a user-defined function.

Preferably, the AI model management subsystem includes an AI model, AI model training, and AI model parameter update.

Preferably, the AI model is a neural network system for generating compounds; the AI model parameters are the parameters of the neural network system; the AI model itself can generate compounds randomly.

Preferably, the filtering conditions include the number of heavy atoms of the compound, the number of hydrogen bond donors, the number of hydrogen bond acceptors, scaffold structure, false positives, and compounds that have been reported in existing patent documents.

Preferably, the data log storage subsystem further includes a function of regulating user permissions.

Correspondingly, the present invention provides a screening method using the drug virtual screening system, which includes the following steps:

Step A: Define the binding characteristics of the ligand in the crystal complex through the analysis of the visualization subsystem. The user downloads the crystal complex structure of the target from the protein crystal structure database, and visualizes the binding position of the ligand in the protein, analyze the binding mode of the ligand and the protein, and extract the features that enhance the affinity of the drug to the protein;

Step B: Input the compounds into the evaluation tool box subsystem, and each compound evaluation module in the evaluation tool box system will output a score, which is then integrated into a comprehensive score through the evaluation function;

Step C: Combine visualization subsystem with the evaluation tool box system to form a complete evaluation pipeline, start the AI model through the AI model management subsystem and start training.

Step D: The large-scale sampling subsystem accepts a sampling quantity parameter input by the user, samples the trained AI model, generates a specified number of compounds, deletes unreasonable and repetitive compounds, and then the user inputs filter conditions to eliminate non-compliant compounds, and the remaining compounds form a compound library;

Step E: The virtual screening subsystem further screens the compounds in the compound library;

Step F: The data log storage subsystem creates and stores the user's log information file when the user uses it to design drugs.

Wherein, the specific steps of step A are: the user downloads the crystal complex structure of the target from the protein crystal structure database, visually view the binding position of the ligand in the protein, analyze the binding mode of the ligand and the protein, and extract the hydrogen bond interaction, hydrophobic interaction and other features that may enhance the affinity of the drug to the protein. The user can assign appropriate weights to each important feature according to the important features of the drug's activity on the interface, and finally integrate it into a pharmacophore evaluation module. When a compound is input to the pharmacophore evaluation module, the evaluation module outputs a score by evaluating the matching degree between the compound and the important feature.

Wherein, the binding characteristics of the ligand can be obtained through the analysis of the visualization subsystem, the binding characteristics of the crystal complexes that have been reported in the relevant literature, or the binding characteristics of the ligands that have been reported in the literature and the analysis of the visualization subsystem.

The compound evaluation module includes: substructure alert, selectivity prediction, activity prediction, structural similarity, molecular weight, number of rotating bonds, number of hydrogen bond donors, number of hydrogen bond acceptors, number of rings, molecular docking score, FEP prediction value, pharmacophore score, lipid-aqueous partition coefficient value, compound toxicity prediction evaluation module.

The compound evaluation module in the evaluation tool box subsystem includes the compound evaluation module of various properties such as the conformational characteristics, physical properties, chemical properties, pharmacokinetic properties, and structural novelty of the compound.

Preferably, in the step C, the AI model outputs the compounds generated by the AI model to the evaluation pipeline through interaction with the evaluation pipeline, collects the scores of the compounds output by the evaluation pipeline, and automatically updates the AI model parameters; after many times repeat of this process, the compound generated by the AI model will get a higher score in the evaluation pipeline; after the AI model training is completed, the AI model parameters are also optimized to suitable values.

Preferably, the step E includes the following steps:

Step E1: Download the protein pdb file of the compound from the pdb library, and preprocess the protein: delete water molecules, hydrogenation, etc., delete irrelevant ligands, and define the pretreatment of the site that needs to be docked;

Step E2: optimize the compound conformation, after generating the 3D conformation of the compound, use the genetic algorithm to search for the conformation with the lowest energy of the compound;

Step E3: docking molecules, sort them in descending order according to the docking score, and select the top 5%-15% compounds;

Step E4: conduct molecular dynamics simulation on the compound selected in Step E3, and screen out qualified compounds from the compound library according to the simulation results.

Preferably, in the evaluation function, a weight is set for each score: w₁, w₂, w₃, . . . wₙ, forming an evaluation function, the evaluation function arithmetic weighted average:

$$\frac{{\sum }_{i = 1}^{n} {w}_{i} {score}_{i}}{{\sum }_{i = 1}^{n} {w}_{i}}$$

or geometric weighted average:

$${\sum }_{i = 1}^{n} {w}_{i} \sqrt{{\prod }_{i = 1}^{n} {score}_{i}^{{w}_{i}}} .$$

The data log storage subsystem, the system will create and store the user's log information file when the user uses the system to design drugs; the log information file records the user's operation records and generates corresponding data;

The data log storage subsystem also includes the function of standardizing user permissions. The system groups users according to different R&D pipelines, and each user has different permissions for data and logs of various projects.

The beneficial effects of the present invention are:

1. Based on the large number of compounds generated by the AI model, the design of the evaluation pipeline is used to make the AI model generate compounds that meet specific needs. Compared with the traditional compound library, the generated compound library has more target specificity.

2. Based on this system, the generation of the compound library is organically combined with the subsequent virtual screening. Users only need to describe the mode of action of the drug on the protein and the requirements for the drug to generate a batch of compounds that meet the expectations. The automated system reduces user intervention and improves the efficiency of research and development.

3. The operation of the user in the system, the defined parameters and the molecules generated by the R&D will all be recorded in the system, which is conducive to the traceability of the R&D. In addition, the system also has strict authority management to ensure data security.

### BRIEF DESCRIPTION OF THE DRAWING

The technical solution of the present application will be further described below with reference to the drawings and embodiments.

FIG. 1 is the functional structure and flow chart of the virtual drug screening system for crystal complexes;

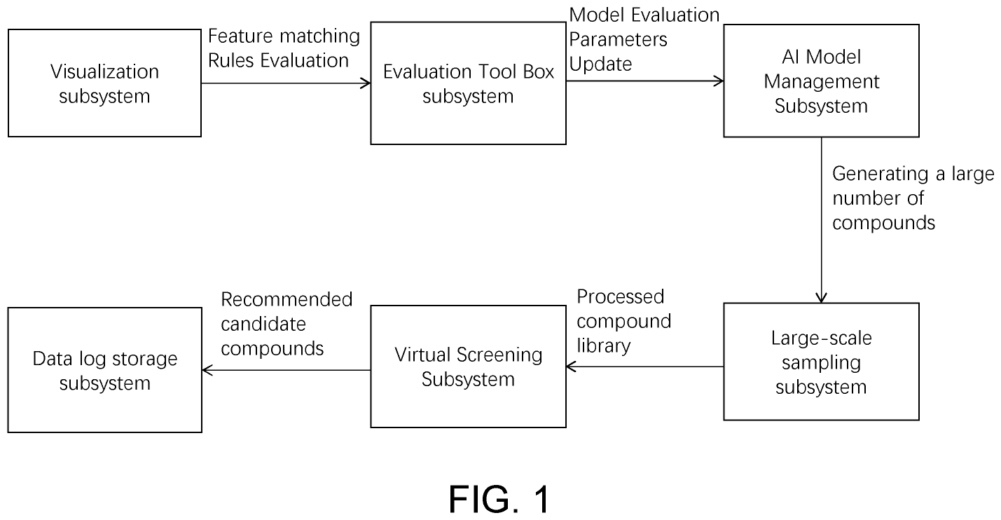

FIG. 2 is a flow chart of the crystal complex drug virtual screening system taking the PARP crystal complex as an example.

FIG. 3 is a schematic diagram of the evaluation pipeline, from a compound input, and finally a final score is returned by the evaluation function.

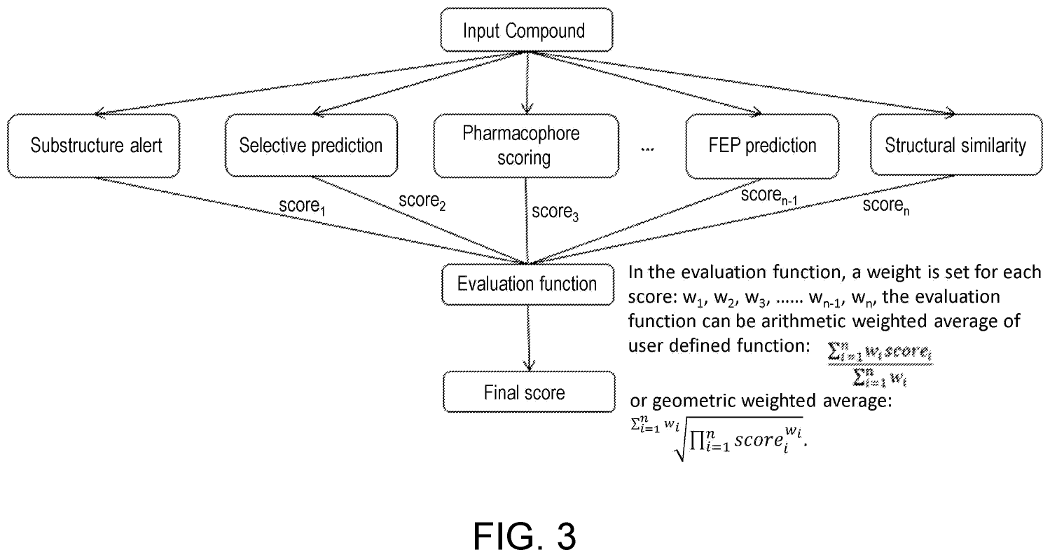

### DESCRIPTION OF THE EMBODIMENTS

### Embodiment 1

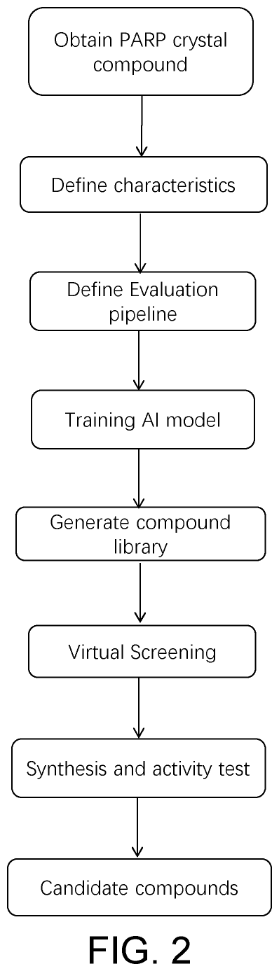

The process shown in FIG. 2:

Polyadenosine diphosphate-ribose polymerase (PARP) participates in the repair of bases by catalyzing the ribosylation of ADP and plays an important role in the repair of single-stranded DNA damage in cells. It is one of the targets of anticancer drugs. PARP1 is a subtype of PARP and one of the targets for the treatment of triple-negative breast cancer. Starting from the crystal complex of PARP1, follow the steps shown in the process (as shown in FIG. 2) to design the drug.

(1) Download the crystal complex structure of PARP1 from the protein crystal structure database. Through the visual analysis of the crystal complex of PARP1, combined with the binding mode reported in the literature, four key pharmacophore characteristics (a hydrogen bond donor characteristic, one hydrogen bond acceptor characteristic, and two hydrophobic characteristics) are determined, and weights are assigned to the four features (the weights are 3, 3, 2, 1 in order) and integrated into a pharmacophore feature evaluation module.

(2) Integrate the key pharmacophore characteristics into a pharmacophore scoring module, and add six modules of substructure alarm, molecular weight, number of rotating bonds, number of hydrogen bond donors, number of hydrogen bond acceptors, and lipid partition coefficient values, and the evaluation function adopts arithmetic weighted average method to form the evaluation pipeline. Except for the weight of the pharmacophore scoring module which is 3, the weights of the other modules are all 1.

(3) Turn on the AI model management subsystem and train the AI model for 1000 rounds.

(4) Input 7 million sampling quantity parameters in the large-scale sampling subsystem, perform large-scale sampling of the AI model, produce more than 7 million compounds, delete unreasonable and repetitive compounds, and finally get more than 800,000 compounds; set the screening conditions to filter the compounds, filter these compounds with physical and chemical properties such as hydrogen bond donors, hydrogen bond acceptors, and the number of heavy atoms, and delete compounds containing substructures such as macrocycles and alkane. Finally, more than 90,000 compounds were obtained.

(5) Search for patents and summarize the known skeletons of PARP inhibitors. Delete compounds with known skeletons to obtain more than 2,000 compounds and form a compound library.

(6) Virtually screen the composed compound library, process the PARP protein and optimize the 3D conformation of the compound, do molecular docking of these compounds, and pick out the top 5% of the scoring compounds for molecular dynamics simulation.

(7) Check and select the conformation of the compound manually, analyze the results of the kinetic simulation, and obtain a batch of candidate compounds.

(8) The system automatically records the user's operation records and candidate compounds generated and sorts and stores them.

### Embodiment 2

Alzheimer's disease is a representative degenerative disease of the central nervous system. Several studies on Alzheimer's disease have found multiple targets in the literature. Acetyl cholinesterase is one of the important targets. Taking the crystal complex of acetyl cholinesterase and its inhibitors as a starting point, look for inhibitors with a new scaffold.

(1) According to literature reports, one of the crystal complexes (PDB: 4EY7) is used as a starting point. Through the visual analysis of the crystal complex (PDB: 4EY7), combined with literature reports, the ligand was located, and 5 key pharmacophore characteristics were determined. These characteristics include 2 hydrogen bond receptors and 2 aromatic ring characteristics, 1 hydrophobic feature; the weight assigned to the pharmacophore feature is 1, integrated into a target feature evaluation module.

(2) Use the pharmacophore model defined in step (1) to combine into a pharmacophore evaluation module, which also supplemented with the two modules of substructure alert and structural similarity. In order to discover new scaffolds, known acetyl cholinesterase inhibitor skeletons were collected from the literature as substructures. Enter these substructures into the substructure alert to determine whether the resulting compound contains the known backbone of the inhibitor. At the same time, the original ligand in the crystal complex is used as the template molecule, and the similarity between the generated molecule and the template molecule is calculated based on the molecular fingerprint. The evaluation function uses arithmetic weighted average to output a final score. Among them, the weight of the pharmacophore scoring module is 5, the weight of the sub-structure alarm module is 10, and the weight of the structural similarity module is 3.

(3) Use the AI model management subsystem to intensively train the AI model for 1000 rounds.

(4) Input 1 million sampling quantity parameters in the large-scale sampling subsystem to generate 1 million compounds. After deleting invalid and repetitive compounds, more than 80,000 compounds were finally obtained. Set the four rules of hydrogen bond donors no more than 5, hydrogen bond acceptors no more than 10, molecular mass less than 500, and lipid-water partition coefficient no more than 5 to filter compounds, eliminate inhibitors containing reported skeletons, and get more than 3,000 remaining compounds to form a compound library.

(5) Conduct molecular docking of more than 3,000 compounds in the compound library, and screen out more than 60 molecules with interactions consistent with literature reports.

(6) The system records the candidate compounds obtained from the screening.

### Embodiment 3

Heat shock protein 90 is a new target of anti-tumor drugs discovered in recent years. Inhibitors of heat shock protein 90 can destroy the structure of the protein in the body and the degradation process to play an anti-tumor effect. After the crystal structure of heat shock protein 90 was published, computer-aided drug design became the mainstream for the development of new heat shock protein 90 inhibitors. This example tried to start with the crystal complex of heat shock protein 90, and recommended a batch of new heat shock protein 90 inhibitors.

(1) Use one of the heat shock protein 90 (PDB: 1YET) as a starting point. Through the visual analysis of heat shock protein 90 (PDB: 1YET), combined with literature reports, define the binding position of the inhibitor on heat shock protein 90 (PDB: 1YET), define 2 hydrogen bond receptors, 2 hydrophobic centers and Two hydrogen bond donors form a pharmacophore model, and the weights of these pharmacophores are 1, integrated into a target feature evaluation module.

(2) Use the pharmacophore model defined in step (1) to combine into a pharmacophore evaluation module, add the molecular weight module, and restrict the molecular weight to be less than 500. In order to be able to evaluate the compound more reasonably, a molecular docking scoring module (using Autodock docking) is connected, and the compound is molecularly docked, and the opposite number of the docking score of the molecular docking is used as the evaluation score. The evaluation function uses arithmetic weighted average to output a final score. Among them, the weight of the pharmacophore scoring module is 3, the weight of the molecular docking scoring module is 5, and the weight of the molecular weight module is 10.

(3) Use the AI model management subsystem to intensively train the AI model for 1000 rounds.

(4) Input the sampling quantity parameter 1 million in the large-scale sampling subsystem, generate 1 million compounds, remove the invalid and repeated compounds, and finally get more than 200,000 compounds, set the number of hydrogen bond donors not to exceed 5. The four rules of acceptor number not exceeding 10, molecular mass lower than 500, and lipid-water partition coefficient not exceeding 5 filter compounds. Inhibitors containing reported skeletons are eliminated, and more than 8,000 compounds are obtained to form a compound library.

(5) Use Tanimoto algorithm to calculate the similarity of compound molecular fingerprints (ECFP4), and find out more than 500 compounds that are most similar to the ligands in the heat shock protein 90 crystal complex from the compound library. More than 30 candidate compounds were screened out using molecular docking and molecular dynamics simulation.

(6) The system records the candidate compounds obtained from the screening.

Taking the above-mentioned ideal embodiments based on this application as enlightenment, through the above description, relevant staff can make various changes and modifications without departing from the scope of the technical idea of this application. The technical scope of this application is not limited to the content in the specification, and its technical scope must be determined according to the scope of the claims.

Those skilled in the art should understand that the embodiments of the present application can be provided as a method, a system, or a computer program product. Therefore, this application may adopt the form of a complete hardware embodiment, a complete software embodiment, or an embodiment combining software and hardware. Moreover, this application may adopt the form of a computer program product implemented on one or more computer-usable storage media (including but not limited to disk storage, CD-ROM, optical storage, etc.) containing computer-usable program codes.

This application is described with reference to the method of embodiments of this invention and flowcharts and/or block diagrams of devices (systems), and computer program products. It should be understood that each process and/or block in the flowchart and/or block diagram, and the combination of processes and/or blocks in the flowchart and/or block diagram can be realized by computer program instructions. These computer program instructions can be provided to the processor of a general-purpose computer, a special-purpose computer, an embedded processor, or other programmable data processing equipment to generate a machine, so that the instructions executed by the processor of the computer or other programmable data processing equipment are generated. It is a device that realizes the functions specified in one process or multiple processes in the flowchart and/or one block or multiple blocks in the block diagram.

These computer program instructions can also be stored in a computer-readable memory that can direct a computer or other programmable data processing equipment to work in a specific manner, so that the instructions stored in the computer-readable memory produce an article of manufacture including the instruction device. The device implements the functions specified in one process or multiple processes in the flowchart and/or one block or multiple blocks in the block diagram.

These computer program instructions can also be loaded on a computer or other programmable data processing equipment, so that a series of operation steps are executed on the computer or other programmable equipment to produce computer-implemented processing, so as to execute on the computer or other programmable equipment. The instructions provide steps for implementing functions specified in a flow or multiple flows in the flowchart and/or a block or multiple blocks in the block diagram.

1. A virtual drug screening system for crystal complexes, comprising: a visualization subsystem, an evaluation tool box subsystem, an AI model management subsystem, a large-scale sampling subsystem, a virtual screening subsystem, and a data log storage subsystem; starting from a known crystal complexes, a batch of candidate compounds that meet the requirements are recommended after sequentially going through the visualization subsystem, the evaluation tool box subsystem, the AI model management subsystem, the large-scale sampling subsystem, and the virtual screening sub system;
wherein the visualization subsystem is used to view the binding position of a ligand of a protein in the crystal complex, analyze a binding mode of the ligand and the protein, and extract features that enhance the affinity of the drug to the protein;
wherein the evaluation tool box subsystem encapsulates a plurality of compound evaluation modules, and is used to design an evaluation function by selecting the plurality of compound evaluation modules and assigning appropriate weights;
wherein the AI model management subsystem is used for AI model, AI model training, and update of AI model parameter; wherein the AI model is a neural network system for generating compounds; the AI model parameter is a parameter of the neural network system; and the AI model itself can generate the compounds randomly;
wherein the large-scale sampling subsystem is used to sample and screen the trained AI model to obtain a compound library composed of the corresponding compounds;
wherein the virtual screening subsystem is used for further screening of the compounds in the compound library;
wherein the data log storage subsystem is used to establish and store a user's log information file; the log information file is used to record user operations and generate corresponding data.

2. The drug virtual screening system according to claim 1, wherein the features that enhance the affinity of the drug to the protein is hydrogen bonding and/or hydrophobic interaction.

3. The drug virtual screening system according to claim 1, wherein the evaluation function is a weighted arithmetic mean, a weighted geometric mean, or a user-defined function.

4. The drug virtual screening system according to claim 1, wherein the AI model management subsystem includes the AI model, the AI model training, and the update of the AI model parameter;
wherein the AI model is a neural network system for generating the compounds;
wherein the AI model parameter is the parameter of the neural network system; and the AI model itself can generate the compounds randomly.

5. The drug virtual screening system according to claim 1, wherein a filter condition of the screening includes a number of heavy atoms of the compound, a number of hydrogen bond donors, a number of hydrogen bond acceptors, scaffold structure, false positives, and the compounds that have been reported in existing patent literature.

6. The drug virtual screening system according to claim 1, wherein the data log storage subsystem further includes a function of standardizing user permissions.

7. A screening method using the drug virtual screening system according to claim 1, comprising following steps of:
Step A: define binding characteristics of the ligand in the crystal complex through an analysis of the visualization subsystem, wherein the user downloads a target of the crystal complex structure from a protein crystal structure database, visualizes a binding position of the ligand in the protein, analyzes the binding mode of the ligand and the protein, and extracts the features that enhance the affinity of the drug to the protein;
Step B: input the compounds into the evaluation tool box subsystem, and each of the plurality of compound evaluation modules in the evaluation tool box subsystem will output a score, which is then integrated into a comprehensive score through the evaluation function;
Step C: combine the visualization subsystem with the evaluation tool box subsystem to form a complete evaluation pipeline, start the AI model through the AI model management subsystem and start the AI model training;
Step D: the large-scale sampling subsystem accepts a sampling quantity parameter input by the user, samples the trained AI model, generates a specified number of compounds, deletes unreasonable and repetitive compounds, and then the user inputs filter conditions to eliminate non-compliant compounds, and the remaining compounds form a compound library;
Step E: the virtual screening subsystem further screens the compounds in the compound library;
Step F: the data log storage subsystem creates and stores the user's log information file when the user uses the subsystem to design drugs.

8. The method according to claim 7, wherein in the Step C, the AI model outputs the compounds generated by the AI model to the evaluation pipeline through interaction, and collects scores of the compounds output by the evaluation pipeline, the AI model parameters are automatically updated; after repeating the Step C for a number of time, the compounds generated by the AI model will get a higher score in the evaluation pipeline; after the AI model training is completed, the AI model parameters are also optimized to suitable values.

9. The method according to claim 7, wherein the Step E comprises following steps of:
protein pretreatment: download a protein PDB file of the compounds from a PDB library, perform protein pretreatment operations, delete water molecules, hydrogenate, delete irrelevant ligands, and define the pretreatment of a site that needs to be docked;
conformation optimization: carry out a conformation optimization operation for the compounds, after generating a 3D conformation of the compounds, use a genetic algorithm to search for the 3D conformation of the compounds in the lowest energy;
molecular docking: perform a molecular docking, sort in descending order according to a score of the molecular docking, and select the compound that having a top 5%-15% of the score;
molecular dynamics simulation: perform molecular dynamics simulation on the selected compounds, and screen out qualified compounds from the compound library based on a result of the molecular dynamics simulation.

10. The method according to claim 7, wherein in the evaluation function, a weight is set for each of the score: w₁, w₂, w₃, . . . wₙ to form the evaluation function, and the evaluation function is an arithmetic weighted average:
$$\frac{{\sum }_{i = 1}^{n} {w}_{i} {score}_{i}}{{\sum }_{i = 1}^{n} {w}_{i}}$$
or a geometric weighted average:
$${\sum }_{i = 1}^{n} {w}_{i} \sqrt{{\prod }_{i = 1}^{n} {score}_{i}^{{w}_{i}}} .$$
